(19) United States
(12) Patent Application Publication
(10) Pub. No.: US 2011/0028422 A1
(43) Pub. Date: Feb. 3, 2011

(54) COMBINATION OF NILOTINIB AND A NITROGEN MUSTARD ANALOGUE FOR THE TREATMENT OF CHRONIC LYMPHOCYTIC LEUKEMIA

(76) Inventors: Raquel Silvia Aloyz, Montreal (CA); Lawrence Carl Panasci, Quebec (CA)

Correspondence Address: NOVARTIS;CORPORATE INTELLECTUAL PROPERTY, ONE HEALTH PLAZA 101/2, EAST HANOVER, NJ, 07936-1080, US

(21) Appl. No.: 12/809,805

(22) PCT Filed: Dec. 18, 2008
(86) PCT No.: PCT/US08/87336

§ 371 Date: Oct. 20, 2010

Related U.S. Application Data
(60) Provisional application No. 61/015,704, filed on Dec. 21, 2007.

(51) Int. Cl. A61K 31/7076; A61K 31/506; A61K 31/567; A61K 31/66; A61K 31/505; A61P 35/02

(52) U.S. Cl. 514/47; 514/275; 514/171; 514/110; 514/274

(57) ABSTRACT

The invention relates to a combination which comprises (a) a DNA damaging agent; and (b) 4-methyl-3-[[4-(3-pyridinyl)-2-pyrimidinyl]amino]-N-[5-(4-methyl-1H-imidazol-1-yl)-3-(trifluoromethyl)phenyl]benzamide (“nilotinib”); a pharmaceutical composition comprising such a combination and optionally at least one pharmaceutically acceptable carrier for simultaneous, separate or sequential use, in particular for the treatment chronic lymphocytic leukemia (CLL); the use of such a combination for the preparation of a medicament for the treatment of CLL; a commercial package or product comprising such a combination; and to a method of treatment of a warm-blooded animal, especially a human.

The invention relates to a combination which comprises (a) a DNA damaging agent; and (b) 4-methyl-3-[[4-(3-pyridinyl)-2-pyrimidinyl]amino]-N-[5-(4-methyl-1H-imidazol-1-yl)-3-(trifluoromethyl)phenyl]benzamide (“nilotinib”); a pharmaceutical composition comprising such a combination and optionally at least one pharmaceutically acceptable carrier for simultaneous, separate or sequential use, in particular for the treatment chronic lymphocytic leukemia (CLL); the use of such a combination for the preparation of a medicament for the treatment of CLL; a commercial package or product comprising such a combination; and to a method of treatment of a warm-blooded animal, especially a human.

Preferably a DNA damaging agent is a nitrogen mustard analogue. Further preferably a nitrogen mustard analogue is selected from a group consisting of chlorambucil, chlornaphazine, estramustine, mechlorethamine, mechlorethamine oxide hydrochloride, navembichin, phenestrine, prednimustine, trofosfamide, uracil mustard, cyclophosphamide, uramustine, melphalan and bendamustine.

Preferably a DNA damaging agent is a nucleotide analogs, such as a purine analogue or a pyrimidine analogue. Preferably a purine analogue is fludarabine.

Chronic lymphocytic leukemia (CLL) is the most frequent form of leukemia in adults accounting for 25% of all leukemias (approximately 10,000 new CLL cases yearly in the United States (US)). In the US, 95% of CLL cases are B-cell phenotype leukemia. About 50% of CLL, patients are asymptomatic at diagnosis. The stage of disease correlates with prognosis; stage O having a median survival of >10 years while stage I-II has a median survival of 7 years. Treatment is usually started when patients are symptomatic.

There are two major groups of drugs utilized in the treatment of CLL: (1) alkylating agents such as chlorambucil (CLB) or cyclophosphamide and (2) purine analogs such as fludarabine. Usually chlorambucil (CLB) was the standard initial therapy, but fludarabine and cyclophosphamide (CTX) have now become the standard front-line treatment. These agents lead to responses in 60-75% of patients. Recent randomized trials demonstrated a higher response rate for fludarabine as compared to CLB but no difference in survival. Either agent is acceptable as front line therapy in CLL. Other agents are available for therapy. Eventually, all patients become resistant to the drugs. There is no therapy capable of curing this disease (Kalil, N. and Cheson, B. D. The Oncologist 4:352-369, 1999). PCT/IB 03/05454 discloses that imatinib mesylate, the active ingredient of Gleevec®, sensitizes B-CLL lymphocytes to CLB. While imatinib in combination with CLB is currently in phase I-II clinical study for the treatment of CLL, it has now surprisingly been found that nilotinib possesses a greater potency than imatinib in sensitizing CLL lymphocytes towards CLB. While not willing to be bound by the theory, we found that both nilotinib and imatinib inhibit in a similar fashion CLB-induced Rad51-related DNA repair, but only nilotinib increased CLB-induced □ H2AX. Analysis of caspase-3 activation showed an increased of the apoptosis pathways mediated by JNK activation in cells treated with CLB in combination with nilotinib, but not imatinib. Moreover, c-abl inhibition by nilotinib leads to down regulation of the NFκB pathway involved in maintenance of survival B-CLL lymphocytes.

FIGURE: Synergistic effect of nilotinib and imatinib in CLB cytotoxicity in lymphocytes from B-CLL patients. Evaluation of the synergistic effect of 1, 5 and 10 μM of nilotinib and imatinib in CLB cytotoxicity was assessed using MTT assay. The I value I<1 indicates that the CLB plus nilotinib or imatinib act synergistically. When I value or I>1 the drugs act antagonistically. *: p<0.001.

The present invention provides a combination for simultaneous, separate or sequential use which comprises (a) DNA-damaging agent and (b) 4-methyl-3-[[4-(3-pyridinyl)-2-pyrimidinyl]amino]-N-[5-(4-methyl-1H-imidazol-1-yl)-3-(trifluoromethyl)phenyl]benzamide, in which the active ingredients (a) and (b) are present in each case in free form or in the form of a pharmaceutically acceptable salt. The present invention further provides said combination for simultaneous, separate or sequential use. Preferably said DNA-damaging agent is a nitrogen mustard analogue.

The present invention provides a combination comprising (a) a nitrogen mustard analogue selected from a group consisting of chlorambucil, chlornaphazine, estramustine, mechlorethamine, mechlorethamine oxide hydrochloride, navembichin, phenestrine, prednimustine, trofosfamide, cyclophosphamide, uramustine, bendamustine, melphalan, and uracil mustard and (b) 4-methyl-3-[[4-(3-pyridinyl)-2-pyrimidinyl]amino]-N-[5-(4-methyl-1H-imidazol-1-yl)-3-(trifluoromethyl)phenyl]benzamide, in which the active ingredients (a) and (b) are present in each case in free form or in the form of a pharmaceutically acceptable salt.

The present invention further provides said combination for simultaneous, separate or sequential use.

Preferably the present invention provides a combination comprising (a) chlorambucil, (b) 4-methyl-3-[[4-(3-pyridinyl)-2-pyrimidinyl]amino]-N-[5-(4-methyl-1H-imidazol-1-yl)-3-(trifluoromethyl)phenyl]benzamide, in which the active ingredients (a) and (b) are present in each case in free form or in the form of a pharmaceutically acceptable salt. The present invention further provides said combination for simultaneous, separate or sequential use.

The present invention provides a combination comprising (a) cyclophosphamide, (b) 4-methyl-3-[[4-(3-pyridinyl)-2-pyrimidinyl]amino]-N-[5-(4-methyl-1H-imidazol-1-yl)-3-(trifluoromethyl)phenyl]benzamide, in which the active ingredients (a) and (b) are present in each case in free form or in the form of a pharmaceutically acceptable salt. The present invention further provides said combination for simultaneous, separate or sequential use.

The present invention provides a combination comprising (a) Bendamustine, (b) 4-methyl-3-[[4-(3-pyridinyl)-2-pyrimidinyl]amino]-N-[5-(4-methyl-1H-imidazol-1-yl)-3-(trifluoromethyl)phenyl]benzamide, in which the active ingredients (a) and (b) are present in each case in free form or in the form of a pharmaceutically acceptable salt. The present invention further provides said combination for simultaneous, separate or sequential use.

The present invention provides a combination comprising (a) fludarabine and (b) 4-methyl-3-[[4-(3-pyridinyl)-2-pyrimidinyl]amino]-N-[5-(4-methyl-1H-imidazol-1-yl)-3-(trifluoromethyl)phenyl]benzamide, in which the active ingredients (a) and (b) are present in each case in free form or in the form of a pharmaceutically acceptable salt. The present invention further provides said combination for simultaneous, separate or sequential use.

The present invention reports that a combination comprising a nitrogen mustard analogue selected from CLB, chlornaphazine, estramustine, mechlorethamine, mechlorethamine oxide hydrochloride, navembichin, phenestrine, prednimustine, trofosfamide or uracil mustard, particularly CLB and nilotinib, can produce a therapeutic effect which is greater than that obtainable by administration of a therapeutically effective amount of either a sole nitrogen mustard analogue, in particular CLB or nilotinib alone. More specifically, nilotinib sensitizes B-CLL lymphocytes to the treatment with CLB.

The present invention pertains to a combination for simultaneous, separate or sequential use, such as a combined preparation or a pharmaceutical fixed combination, which comprises (a) a nitrogen mustard analogue and (b) nilotinib in which the active ingredients (a) and (b) are present in each case in free form or in the form of a pharmaceutically acceptable salt, and optionally at least one pharmaceutically acceptable carrier.

The term “a combined preparation”, as used herein defines especially a “kit of parts” in the sense that the combination partners (a) and (b) as defined above can be dosed independently of each other or by use of different fixed combinations with distinguished amounts of the combination partners (a) and (b), i.e., simultaneously or at different time points. The parts of the kit of parts can then, e.g., be administered simultaneously or chronologically staggered, that is at different time points and with equal or different time intervals for any part of the kit of parts. Very preferably, the time intervals are chosen such that the effect on the treated disease in the combined use of the parts is larger than the effect which would be obtained by use of only any one of the combination partners (a) and (b). The ratio of the total amounts of the combination partner (a) to the combination partner (b) to be administered in the combined preparation can be varied, e.g. in order to cope with the needs of a patient sub-population to be treated or the needs of the single patient which different needs can be due to age, sex, body weight, etc. of the patients. Preferably, there is at least one beneficial effect, e.g., a mutual enhancing of the effect of the combination partners (a) and (b), in particular a synergism, e.g. a more than additive effect, additional advantageous effects, less side effects, a combined therapeutic effect in a non-effective dosage of one or both of the combination partners (a) and (b), and very preferably a strong synergism of the combination partners (a) and (b).

The term “treatment” comprises the administration of the combination partners to a warm-blooded animal in need of such treatment with the aim to cure the disease or to effect a delay of progression of a disease.

The term “delay of progression” as used herein means that the disease progression is at least slowed down or hampered by the treatment and that patients exhibit higher survival rates than patients not being treated or being treated with the monotherapy.

By “nitrogen mustard analogue”, as commonly understood by a skilled person, refers to a cytotoxic chemotherapy agent which non-specifically alkylates DNA. Preferably the term “nitrogen mustard analogue” refers to a group of compounds, including but not limited to CLB, chiornaphazine, estramustine, mechlorethamine, mechlorethamine oxide hydrochloride, navembichin, phenestrine, prednimustine, trofosfamide, uracil mustard cyclophosphamide, uramustine, bendamustine and melphalan.

The term “chlorambucil-resistant chronic lymphocytic leukemia” as used herein defines especially a chronic lymphocytic leukemia in which CLB is no longer efficient or shows a reduction of its therapeutic effectiveness.

CLB can be prepared according to the process described in U.S. Pat. No. 3,046,301.

4-Methyl-3-[[4-(3-pyridinyl)-2-pyrimidinyl]amino]-N-[5-(4-methyl-1H-imidazol-1-yl)-3-(trifluoromethyl)phenyl]benzamide is also known under the international non-proprietary name “nilotinib”. It can be prepared and administered as described in WO 04/005281.

Nilotinib can be employed in the form of its mono-hydrochloride mono-hydrate as disclosed in WO2007/015870.

The structure of the active agents cited may be taken from the actual edition of the standard compendium “The Merck Index” or from databases, e.g. Patents International (e.g. IMS World Publications). The corresponding content thereof is hereby incorporated by reference. Any person skilled in the art is fully enable, based on these references, to manufacture and test the pharmaceutical indications and properties in standard test models, both in vitro and in vivo.

A combination as disclosed in the present invention, for example a combination which comprises (a) a nitrogen mustard analogue, preferably CLB or cyclophosphamide, or alternatively (a) fludarabine and (b) nilotinib in which the active ingredients are present in each case in free form or in the form of a pharmaceutically acceptable salt and optionally at least one pharmaceutically acceptable carrier, will be referred to hereinafter as a COMBINATION OF THE INVENTION.

The COMBINATIONS OF THE INVENTION exhibit beneficial effects in the treatment of CLL. In one preferred embodiment of the invention, the proliferative disease to be treated with a COMBINATION OF THE INVENTION is CLL, which is resistant to CLB.

Surprisingly, the COMBINATIONS OF THE INVENTION is also better tolerated by CLL patients than the corresponding combinations employing imatinib mesylate instead of nilotinib or a pharmaceutically acceptable salt thereof.

It can be shown by established test models that a COMBINATION OF THE INVENTION results in the beneficial effects described herein before. The person skilled in the pertinent art is fully enabled to select a relevant test model to prove such beneficial effects. The pharmacological activity of a COMBINATION OF THE INVENTION may, for example, be demonstrated in a clinical study or in a test procedure as essentially described hereinafter.

Suitable clinical studies are in particular randomized, double-blind, parallel studies in CLL patients with late stage disease. Such studies are, in particular, suitable to compare the effects of a mono-therapy using the active ingredients independent of each other and a therapy using a COMBINATION OF THE INVENTION, and to prove in particular the synergism of the active ingredients of the COMBINATIONS OF THE INVENTION. The primary endpoints in such studies can be the performance status, Quality of Life scores or time to progression of the disease. In a suitable study design, patients are, for example, receiving per treatment cycle of 2 weeks, daily at a dose ranging from 50 to 1000 mg of the nilotinib and CLB at a dose ranging from 0.2 to 1 mg/kg/day.

It is one objective of this invention to provide a pharmaceutical composition comprising a quantity, which is jointly therapeutically effective against CLL comprising the COMBINATION OF THE INVENTION. In this composition, the combination partners (a) and (b) can be administered together, one after the other or separately in one combined unit dosage form or in two separate unit dosage forms. The unit dosage form may also be a fixed combination.

The pharmaceutical compositions according to the invention can be prepared in a manner known per se and are those suitable for enteral, such as oral or rectal, and parenteral administration to mammals (warm-blooded animals), including man, comprising at least one pharmacologically active combination partner alone or in combination with one or more pharmaceutically acceptable carries, especially suitable for enteral or parenteral application. In one embodiment of the invention, one or more of the active ingredients are administered orally.

In particular, a therapeutically effective amount of each of the combination partners of the COMBINATION OF THE INVENTION may be administered simultaneously or sequentially and in any order, and the components may be administered separately or as a fixed combination. For example, a method for delaying the progression or treatment of CLL according to the present invention may comprise (i) administration of the first combination partner in free or pharmaceutically acceptable salt form and (ii) administration of the second combination partner in free or pharmaceutically acceptable salt form, simultaneously or sequentially in any order, in jointly therapeutically effective amounts, preferably in synergistically effective amounts, e.g. in daily dosages corresponding to the amounts described herein. The individual combination partners of the COMBINATION OF THE INVENTION can be administered separately at different times during the course of therapy or concurrently in divided or single combination forms. Furthermore, the term administering also encompasses the use of a pro-drug of a combination partner that convert in vivo to the combination partner as such. The instant invention is therefore to be understood as embracing all such regimes of simultaneous or alternating treatment and the term “administering” is to be interpreted accordingly.

The effective dosage of each of the combination partners employed in the COMBINATION OF THE INVENTION may vary depending on the particular compound or pharmaceutical composition employed, the mode of administration, the stage of CLL being treated and the severity of the condition being treated. Thus, the dosage regimen the COMBINATION OF THE INVENTION is selected in accordance with a variety of factors including the route of administration and the renal and hepatic function of the patient. A physician, clinician or veterinarian of ordinary skill can readily determine and prescribe the effective amount of the single active ingredients required to prevent, counter or arrest the progress of the condition. Optimal precision in achieving concentration of the active ingredients within the range that yields efficacy without toxicity requires a regimen based on the kinetics of the active ingredients' availability to target sites. This involves a consideration of the distribution, equilibrium, and elimination of the active ingredients.

CLB can be administered at a dose range of 0.1 to 1 mg/kg/day, preferably at a dose range of 0.2 to 0.8 mg/kg/day. Most preferably, CLB is administered at a dose of 0.6 mg/kg/day.

Depending on species, age, individual condition, mode of administration, and the clinical picture in question, daily doses of about 50 to 1000 mg of nilotinib are administered to warm-blooded animals of about 70 kg bodyweight.

The invention relates also to a method for administering to a human subject suffering from CLL nilotinib or a pharmaceutically acceptable salt thereof.

When the combination partners employed in the COMBINATION OF THE INVENTION are applied in the form as marketed as single drugs, their dosage and mode of administration can take place in accordance with the information provided on the packet leaflet of the respective marketed drug in order to result in the beneficial effect described herein, if not mentioned herein otherwise.

The COMBINATION OF THE INVENTION can be a combined preparation or a pharmaceutical composition.

Furthermore, the present invention pertains to the use of a COMBINATION OF THE INVENTION for the treatment of CLL and for the preparation of a medicament for the treatment of CLL.

Additionally, the present invention pertains to the use of CLB in combination with nilotinib for the preparation of a medicament for the treatment of CLL.

Moreover, the present invention provides a commercial package comprising as active ingredients COMBINATION OF THE INVENTION, together with instructions for simultaneous, separate or sequential use thereof in the treatment of CLL.

### EXAMPLE I

### Nilotinib Sensitizes CLL Lymphocytes to CLB

### A-Material and Methods

A-1) Isolation of CLL Lymphocytes and cell culture Lymphocytes are isolated from the peripheral blood of CLL patients by sedimentation centrifugation on Ficoll Hypaque (Pharmacia, Uppsala, Sweden) as described previously (Christodoulopolis G. et al., Cancer Research, 1999, 5:2178-84). Aliquots containing 1×10⁶ cells/ml are sent for T-lymphocyte analysis. The percentage of contaminating T lymphocytes is determined using fluorescence-activated cell sorting analysis with CD3 antibody. The percentage of T-lymphocyte contamination in our population (expressed as a mean %±SE) is 6.4±1.8. The WSU cell line is a B-lymphocytic cell line derived from a CLL patient (Mohammad R. M., et al., Leukemia, 1996, 10:130-7).

A-2) Plating Efficiency and Dosing. The lymphocytes and WSU CLL lymphocytes are seeded into 96-well plates in 200 μl suspensions containing 1.5×10⁶ lymphocytes/ml and 1.25×10⁵ cells/ml respectively in RPMI supplemented with 10% FBS. Only dose responses with linear plating efficiencies are analyzed. The lymphocytes are then incubated at 37° C. in the presence of various concentrations of nilotinib (0-100 μM) alone, CLB (0-100 μM) alone, or various concentrations of both drugs together.

A-3) Cytotoxic Assay. The MIT assay is performed 72 hours after plating as described before (Christodoulopolis G et al., Cancer Research, 1998, 58:1789-92) by addition of 20 μl of a solution of 5 mg/ml MTT (3-[4,5-dimethylthiazol-2-yl]2,5-diphenyl-tetrazolium bromide) in RPMI media to each well. The LD₅₀ of CLB alone, nilotinib alone or CLB in the presence of nilotinib is defined as to be the drug concentration required to reduce the absorbance reading to 50% of the control value. The percentage of surviving cells after treatment respect to vehicle treated cells (control) is calculated as (OD treated cells/OD untreated cells)×100. Synergy is determined by the formula: a/A+b/B=1 where a is the concentration of CLB required to produce 50% of control values in combination with nilotinib at concentration b; A is the concentration of CLB that produces an LD₅₀ without nilotinib; and B is the concentration of nilotinib that produces an LD₅₀ in the absence of CLB. According to the formula, when I<1 the interaction is synergistic, when I=1, the interaction is additive, and when I>1 there is an antagonistic interaction.

A-4) Statistical Analysis Differences between mean values is assessed by two tailed t-test. Correlation and linear regression analysis are performed using the EXCEL Statistical Tool Package.

### Results

Nilotinib possessed greater potency than imatinib in sensitizing CLL lymphocytes (16/19 samples) towards chlorambucil.

### EXAMPLE II

Patients. Twenty one patients with a diagnosis of B-CLL followed at the Jewish General Hospital of Montreal were enrolled in the study after informed consent. Patients were either clinically untreated (n=13) or treated with CLB (n=8) for various time periods.

Cytotoxicity assay. Lymphocytes were isolated from the peripheral blood using Ficoll-Hypaque (Pharmacia). The T-lymphocyte contamination in the isolated B-lymphocytes population was 2.30±2.07 (expressed as a mean %±S.D. and determined by flow cytometry analysis). The CLL lymphocytes (3×10⁶ cells/ml) were plated in RPMI 1640 supplemented with 10% FBS and incubated in the presence of various concentrations (0-100 μM) of imatinib (Novartis), nilotinib (Novartis), chlorambucil alone (Sigma-Aldrich Co), or in combination as indicated. Control samples were incubated with the greatest volume of DMSO. The MTT assay was performed 72 h after treatment as previously described (Christodoulopoulos et al, Cancer Res, 58: 1789-1792, 199835). Synergy was determined by the formula: a/A+b/B=I, where a is the CLB IC₅₀ (concentration resulting in 50% of control) in combination with imatinib or nilotinib at concentration b; A is the CLB IC₅₀ without imatinib or nilotinib; and B is the imatinib or nilotinib IC₅₀ in the absence of CLB. According to the formula, when I<1, the interaction is synergistic, when I=1, the interaction is additive, and when I>1 there is an antagonistic interactio.

Statistical analysis. Statistical analysis by ANOVA and t-test were done with SigmaStat software (Systat Software Inc., San Jose, Calif., USA).

### Results

Both c-abl Inhibitors Sensitized B-CLL Lymphocytes to Chlorambucil

A total of 21 CLL patients were enrolled on this study. Thirteen patients were clinically untreated (patients 1 to 13) and eight had received prior therapy with chlorambucil (treated patients 14 to 21). Their median age was 72 years (range 45 to 90 years) and their median WBC count was 83.7×10⁹ cells/liter (range 32.87×10⁹ to 256.27×10⁹ cells/liter).

The MTT assay was utilized to determine the cytotoxicity of CLB alone, imatinib alone, nilotinib alone or the combination of CLB with 1, 5 or 10 μM imatinib or nilotinib in lymphocytes from CLL patients.

When used at 1, 5 or 10 μM, nilotinib sensitized (synergistic plus additive effect) CLL lymphocytes to CLB in 84.6%, 100% and 90.5% of the patients tested respectively, while imatinib sensitized only 66.7%, 68.4% and 36.8% of these samples to CLB at similar concentrations. Interestingly, sensitization of B-CLL lymphocytes to CLB is statistically more potent with 5 or 10 μM nilotinib than with imatinib (FIGURE).

Furthermore, Five out of the twenty one patients were selected in their ability to sensitize B-CLL lymphocytes to CLB. Using the MTT assay, we evaluated the effect of nilotinib and imatinib on CLB cytotoxicity in malignant B lymphocytes from these five patients. The I-value, I<1 or I>1, indicates that the CLB and c-abl inhibitors act synergistically or antagonistically, respectively.

TABLE

| | | | | CLB + 5 μM | | CLB + 5 μM | |
|---|---|---|---|---|---|---|---|
| | CLB | Nilotinib | Imatinib | nilotinib | Synergy | imatinib | Synergy |
| Patient | IC₅₀ (μM) | IC₅₀ (μM) | IC₅₀ (μM) | IC₅₀ (μM) | Value I | IC₅₀ (μM) | Value I |
| 9 | 11.68 | >100 | 43.32 | 8.19 | 0.70 | 12.43 | 1.18 |
| 14 | 8.10 | 33.16 | 42.07 | <2.5 | 0.38 | 6.07 | 0.87 |
| 15 | 49.69 | 19.92 | 37.92 | 41.06 | 1.08 | 66.60 | 1.47 |
| 16 | 4.54 | 9.61 | 14.22 | <2.5 | 0.68 | 3.51 | 1.12 |
| 18 | 66.02 | 28.73 | 38.35 | 37.13 | 0.69 | 55.56 | 1.00 |

Conclusion: We now demonstrate that nilotinib, a more potent c-abl inhibitor than imatinib, has a greater efficacy to synergize CLB cytotoxicity in B-CLL lymphocytes.

1. A combination for simultaneous, separate or sequential use which comprises (a) DNA-damaging agent and (b) 4-methyl-3-[[4-(3-pyridinyl)-2-pyrimidinyl]amino]-N-[5-(4-methyl-1H-imidazol-1-yl)-3-(trifluoromethyl)phenyl]benzamide, in which the active ingredients (a) and (b) are present in each case in free form or in the form of a pharmaceutically acceptable salt.

2. The combination of claim 1, wherein said DNA-damaging agent is a nitrogen mustard analogue.

3. The combination of claim 2, wherein said nitrogen mustard analogue selected from chlorambucil, chlornaphazine, estramustine, mechlorethamine, mechlorethamine oxide hydrochloride, navembichin, phenestrine, prednimustine, trofosfamide, cyclophosphamide, uramustine and melphalan and uracil mustard.

4. The combination according to claim 3 wherein the nitrogen mustard analogue is chlorambucil.

5. The combination of claim 3, wherein said nitrogen mustard analogue is cyclophosphamide.

6. The combination of claim 3, wherein said nitrogen mustard analogue is bendamustine.

7. The combination of claim 1, wherein said DNA-damaging agent is fludarabine.

8. The combination according to claim 1, wherein compound (b) is used in the form of its mono-hydrochloride mono-hydrate.

9-11. (canceled)

12. A method of treating a warm-blooded animal having chronic lymphocytic leukemia comprising administering to said animal a combination according to claim 1 in a quantity which is jointly therapeutically effective against said disease and in which the compounds can also be present in the form of their pharmaceutically acceptable salts.

13. The method according to claim 12 wherein (a) chlorambucil is administered at a dose of 0.2 to 0.8 mg/kg of body weight/day.

14. A pharmaceutical composition comprising a combination according to claim 1 and at least one pharmaceutically acceptable carrier.

15. A commercial package comprising a pharmaceutical composition according to claim 14 together with instructions for simultaneous, separate or sequential use thereof in the treatment of chronic lymphocytic leukemia.
